(19) United States
(12) Patent Application Publication
(10) Pub. No.: US 2026/0076909 A1
(43) Pub. Date: Mar. 19, 2026

(54) SOLID COMPOSITION AND METHOD FOR PRODUCING THE SAME

(71) Applicant: DKS Co. Ltd., Kyoto-shi, Kyoto (JP)

(72) Inventors: Toru IWAKI, Kyoto-shi (JP); Yuri KADO, Kyoto-shi (JP); Kohei MATSUMOTO, Kyoto-shi (JP); YUKO MORITA, Kyoto-shi (JP); Kaito ONISHI, Kyoto-shi (JP); Mai WATANABE, Kyoto-shi (JP); Kana TERAGAMI, Kyoto-shi (JP)

(73) Assignee: DKS Co. Ltd., Kyoto-shi, Kyoto (JP)

(21) Appl. No.: 19/109,989

(22) PCT Filed: Oct. 12, 2023
(86) PCT No.: PCT/JP2023/036988

§ 371 Date: Mar. 7, 2025

(30) Foreign Application Priority Data
Oct. 21, 2022 (JP) 2022-168943
Sep. 20, 2023 (JP) 2023-152496

(51) Int. Cl. A61K 9/14; A61K 9/00; A61K 31/00

(52) U.S. Cl. CPC A61K 9/145; A61K 9/0053; A61K 9/146; A61K 31/658

(57) ABSTRACT

The solubility of cannabinoid in water is improved.

A solid composition according to an embodiment contains an amorphous cannabinoid, a sucrose fatty acid ester having a monoester ratio of 85 mass % or more, and a water-soluble polymer. The solid composition can be prepared by, for example, dissolving a cannabinoid, a sucrose fatty acid ester having a monoester ratio of 85 mass % or more, and a water-soluble polymer in a solvent, and removing the solvent from the resultant solution.

### TECHNICAL FIELD

Embodiments according to the present invention relate to a solid composition including a cannabinoid and a method for producing the solid composition.

### BACKGROUND ART

Cannabinoids are contained in leaves and seed coats of Cannabis sativa (cannabis). Such a cannabinoid, for example, cannabidiol (CBD) has effects such as control of homeostasis of the body and hence is expected to be applied to the medical field including pharmaceuticals and the food field including food additives.

However, cannabinoids such as cannabidiol are slightly water-soluble and have low efficiency of absorption into the body through oral ingestion. In order to increase the absorption efficiency, the solubility of cannabinoids in water needs to be increased.

A known method for increasing the solubility of slightly water-soluble medicaments in water is to turn the slightly water-soluble medicaments from the crystalline state to the amorphous state. For example, Patent Literature 1 discloses a solid composition containing an amorphous slightly water-soluble polyphenol, a hydrophilic polymer, and a nonionic surfactant, and states that the nonionic surfactant employed is a sucrose fatty acid ester having an HLB value of 10 or more.

Note that Patent Literature 2 describes a method for producing, by a spray drying method, a cannabidiol powder having improved dispersibility of cannabidiol in water. However, in Patent Literature 2, cannabidiol oil together with a sucrose fatty acid ester is dissolved in ethanol; the ethanol solution is mixed with an aqueous solution of a water-soluble polymer such as modified starch to achieve emulsification; and the resultant emulsion is spray-dried and turned into powder. This is a technique relating to powdered oils and is different from solid dispersion techniques of amorphizing slightly water-soluble medicaments. In the technique for powdered oils, an emulsifier is used to turn oil, together with an excipient and the like, into an emulsion in water, to produce an oil-in-water type (O/W type) emulsion; and the emulsion is spray-dried and turned into powder. In the obtained powder, the oil is present as small oil droplets surrounded by the excipient such as glucide or protein, and can be re-dispersed in water as oil droplets (refer to Non Patent Literature 1). In other words, in the powder obtained in Patent Literature 2, the cannabidiol is present as oil droplets (namely, cannabidiol oil) within the solid water-soluble polymer serving as the excipient; therefore, Patent Literature 2 does not describe amorphous cannabinoid.

### CITATION LIST

### Patent Literature

PTL 1: International Publication No. 2017/061627
PTL 2: Chinese Patent Application Publication No. 112891310

### Non Patent Literature

NPL 1: Hironori I N O, “The Characteristic of Powdered Oils and Its Application to Food”, Journal of Oleo Science, vol. 19, No. 10 (2019) p 417-422, Japan Oil Chemists' Society

### SUMMARY OF INVENTION

### Technical Problem

An object of embodiments according to the present invention is to provide a solid composition in which a cannabinoid can have improved solubility in water.

### Solution to Problem

The present invention includes the following embodiments.

[1] A solid composition containing an amorphous cannabinoid, a sucrose fatty acid ester having a monoester ratio of 85 mass % or more, and a water-soluble polymer.

[2] The solid composition according to [1], wherein a mass ratio of the sucrose fatty acid ester to the cannabinoid is 0.2 to 30.

[3] The solid composition according to [1] or [2], wherein a mass ratio of the water-soluble polymer to the cannabinoid is 0.2 to 30.

[4] The solid composition according to any one of [1] to [3], wherein a mass ratio of the water-soluble polymer to the sucrose fatty acid ester is 0.1 to 10.

[5] The solid composition according to any one of [1] to [4], wherein the sucrose fatty acid ester includes a fatty acid having 12 to 22 carbon atoms as a constituent fatty acid.

[6] The solid composition according to any one of [1] to [5], wherein the water-soluble polymer includes at least one selected from the group consisting of homopolymers of N-vinyllactams, copolymers of N-vinyllactams, cellulose ethers, cellulose esters, polyalkylene glycols, and polyalkylene oxides.

[7] The solid composition according to any one of [1] to [6], wherein the cannabinoid includes at least one selected from the group consisting of cannabidiol, cannabigerol, tetrahydrocannabinol, and cannabinol.

[8] An oral composition including the solid composition according to any one of [1] to [7].

[9] A method for producing a solid composition, the method including dissolving a cannabinoid, a sucrose fatty acid ester having a monoester ratio of 85 mass % or more, and a water-soluble polymer in a solvent to obtain a solution, and removing the solvent from the solution.

### Advantageous Effects of Invention

Embodiments of the present invention can provide a solid composition in which a cannabinoid can have improved solubility in water.

### BRIEF DESCRIPTION OF DRAWINGS

FIG. 1 is an XRD chart of a solid composition of Example 3.

FIG. 2 is an XRD chart of a solid composition of Example 5.

FIG. 3 is an XRD chart of a solid composition of Example 6.

FIG. 4 is an XRD chart of a solid composition of Comparative Example 1.

FIG. 5 is an XRD chart of a solid composition of Comparative Example 2.

FIG. 6 is an XRD chart of a solid composition of Comparative Example 4.

### DESCRIPTION OF EMBODIMENTS

A solid composition according to an embodiment contains (A) an amorphous cannabinoid, (B) a sucrose fatty acid ester, and (C) a water-soluble polymer.

### [(A) Amorphous Cannabinoid]

The amorphous cannabinoid refers to a cannabinoid, which is, in general, crystalline and hence is slightly water-soluble, the cannabinoid having been amorphized. The cannabinoid employed can be, for example, the group of various compounds included in the cannabinoid derived from Cannabis sativa, and may be an acidic cannabinoid having a carboxy group, or a neutral cannabinoid not having carboxy groups. In one embodiment, a cannabinoid not having carboxy groups, but having one or two hydroxy groups may be employed. Specific examples include cannabidiol (CBD), cannabigerol (CBG), tetrahydrocannabinol (THC), cannabinol (CBN), cannabichromene (CBC), cannabielsoin (CBE), and cannabidivarin (CBDV), and one or two or more of the foregoing can be employed.

In one embodiment, the cannabinoid preferably includes at least one selected from the group consisting of cannabidiol, cannabigerol, tetrahydrocannabinol, and cannabinol; in this case, the content of the at least one relative to 100 mass % of the cannabinoid is preferably 50 to 100 mass %, more preferably 80 to 100 mass %, more preferably 90 to 100 mass %, still more preferably 95 to 100 mass %.

In one embodiment, the cannabinoid preferably includes cannabidiol and/or cannabigerol. The cannabidiol is known to have the sedative effect, the stress reduction effect, and the like, but to have no addictiveness, and may be applied to the medical field including pharmaceuticals and the food field including food additives. The cannabigerol is known to have the antimicrobial effect, the analgesic effect, the antidepressant effect, and the like, and may be applied to the medical field including pharmaceuticals and the food field including food additives. The content of cannabidiol and/or cannabigerol relative to 100 mass % of the cannabinoid is preferably 50 to 100 mass %, more preferably 80 to 100 mass %, more preferably 90 to 100 mass %, still more preferably 95 to 100 mass % or more, or may be 100 mass % (in other words, the cannabinoid is composed only of cannabidiol and/or cannabigerol).

The cannabinoid employed may be a cannabinoid extracted from Cannabis sativa or may be a cannabinoid chemically synthesized. Similarly, the cannabidiol and/or cannabigerol employed may be a cannabidiol and/or a cannabigerol extracted from Cannabis sativa or may be a cannabidiol and/or a cannabigerol chemically synthesized.

The solid composition according to the embodiment includes, as the cannabinoid, an amorphous cannabinoid. The amorphous refers to being in a noncrystalline, solid form (state). The cannabinoid can be determined as having the amorphous form by X-ray diffractometry (XRD) or differential scanning calorimetry (DSC).

The solid composition may contain, in addition to the amorphous cannabinoid, a crystalline cannabinoid. However, the amount of the crystalline cannabinoid is preferably as small as possible, and preferably no crystalline cannabinoid is substantially or completely contained. In one embodiment, preferably 80 mass % or more, more preferably 90 mass % or more, more preferably 95 mass % or more, more preferably 98 mass % or more, more preferably the entirety of the cannabinoid included in the solid composition is present in the amorphous form.

In the solid composition, the content of the amorphous cannabinoid is not particularly limited, but is preferably 1 to 50 mass %, more preferably 3 to 40 mass %, more preferably 4 to 40 mass %, more preferably 5 to 30 mass %, still more preferably 6 to 20 mass %.

### [(B) Sucrose Fatty Acid Ester]

In the embodiment, a sucrose fatty acid ester having a monoester ratio of 85 mass % or more is employed. In the sucrose fatty acid ester, a fatty acid is ester-bonded to a hydroxy group of sucrose. A molecule of sucrose has eight hydroxy groups and, depending on the number of hydroxy groups to which fatty acids are ester-bonded, a monoester to an octaester are provided. In the embodiment, in the sucrose fatty acid ester employed, the ratio of the monoester in which a fatty acid is bonded to a hydroxy group relative to 100 mass % of the sucrose fatty acid ester is 85 mass % or more. While ordinary sucrose fatty acid esters have a monoester ratio of 80 mass % or less, the sucrose fatty acid ester employed in the embodiment has a high monoester ratio of 85 mass % or more. This can provide improved solubility of the cannabinoid in water.

The monoester ratio in the sucrose fatty acid ester is preferably 90 mass % or more, more preferably 95 mass % or more, still more preferably 97 mass % or more, still more preferably 99 mass % or more, or may be 100 mass %. Thus, the ester ratio of di- or higher esters is preferably 10 mass % or less, more preferably 5 mass % or less, still more preferably 3 mass % or less, still more preferably 1 mass % or less.

In the sucrose fatty acid ester, the monoester ratio can be determined by performing GPC (gel permeation chromatography) and analyzing the ester distribution of the sucrose fatty acid ester, and can be determined as the ratio of the peak area derived from the monoester to the total peak area. The analysis conditions of GPC are as follows.

### <GPC Conditions>

Apparatus: “LC-6A” manufactured by SHIMADZU CORPORATION
Column: “Megapak GEL201” manufactured by JASCO Corporation
Solvent: THF
Flow rate: 3 mL/min
Sample concentration: 6 w/v %
Sample injection amount: 50 μL
Column temperature: 25° C.

The sucrose fatty acid ester preferably includes, as a constituent fatty acid, a fatty acid having 12 to 22 carbon atoms. In other words, as the fatty acid constituting the sucrose fatty acid ester, a fatty acid that has 12 to 22 carbon atoms, is saturated or unsaturated, and is linear or branched is preferably employed; one or a combination of two or more of such fatty acids can be employed. The constituent fatty acid preferably includes a fatty acid having 12 to 22 carbon atoms as a main component, and more preferably includes, as a main component, a fatty acid having 14 to 18 carbon atoms, still more preferably 16 to 18 carbon atoms. The main component means that the content relative to 100 mass % of the constituent fatty acid is 50 mass % or more, more preferably 70 mass % or more, more preferably 80 mass % or more, still more preferably 90 mass % or more, or may be 100 mass %.

The constituent fatty acid of the sucrose fatty acid ester is preferably a saturated fatty acid, and preferably a linear saturated fatty acid. In one preferred embodiment, the constituent fatty acid contains stearic acid as a main component and a mixture of stearic acid and palmitic acid is preferably employed.

The HLB value of the sucrose fatty acid ester is not particularly limited, may be 10 or more, may be 13 or more, or may be 16 or more.

### [(C) Water-Soluble Polymer]

The water-soluble polymer is a natural or synthetic polymer having a property of being soluble in water. For the solubility of the water-soluble polymer in water, the solubility (maximum dissolution concentration) at 25° C. in pure water is preferably 0.001 mass % or more, more preferably 0.1 mass % or more, or may be 1 mass % or more.

Specific examples of the water-soluble polymer include homopolymers of N-vinyllactams and copolymers of N-vinyllactams such as polyvinylpyrrolidone (PVP), copovidone (specifically, a copolymer of N-vinylpyrrolidone and vinyl acetate), and a copolymer of N-vinylpyrrolidone and vinyl propionate; cellulose ethers such as alkylcelluloses (such as methylcellulose and ethylcellulose), hydroxyalkylcelluloses (such as hydroxypropylcellulose (HPC)), and hydroxyalkyl alkylcelluloses (such as hydroxypropyl methylcellulose (HPMC) and hydroxyethyl methylcellulose (HEMC)); cellulose esters such as cellulose phthalate, cellulose acetate phthalate, hydroxypropyl methylcellulose phthalate (HPMCP), hydroxypropyl methylcellulose succinate, and hydroxypropyl methylcellulose acetate succinate (HPMCAS); polyalkylene glycols (PAG) having a structure corresponding to a polymer of an alkylene glycol having 2 to 4 carbon atoms such as polyethylene glycol (PEG) and polypropylene glycol (PPG); polyalkylene oxides (PAO) such as polyethylene oxide (PEO), polypropylene oxide (PPO), and a copolymer of ethylene oxide and propylene oxide; poly(meth)acrylates such as a methacrylic acid/ethyl acrylate copolymer, a methacrylic acid/methyl methacrylate copolymer, a butyl methacrylate/2-dimethylaminoethyl methacrylate copolymer, poly(hydroxyalkyl acrylate), and poly(hydroxyalkyl methacrylate); polyacrylamide; polyvinyl alcohol; and oligosaccharides and polysaccharides such as carrageenan, galactomannan, xanthan gum, and gum arabic. These may be used alone or in combination of two or more thereof. Note that the polyalkylene glycols and the polyalkylene oxides are compounds basically having the same structure; for the former, compounds having an average molecular weight of about 20000 or less and known to those skilled in the art are used and, for the latter, compounds having a higher molecular weight and known to those skilled in the art are used.

Of these, the water-soluble polymer employed is preferably at least one (C1) selected from the group consisting of homopolymers of N-vinyllactams and copolymers of N-vinyllactams, cellulose ethers, cellulose esters, polyalkylene glycols, and polyalkylene oxides. In this case, the content of the at least one (C1) relative to 100 mass % of the water-soluble polymer is preferably 70 to 100 mass %, more preferably 80 to 100 mass %, more preferably 90 to 100 mass %, still more preferably 95 to 100 mass %.

In one embodiment, the water-soluble polymer preferably includes at least one (C2) selected from the group consisting of polyvinylpyrrolidone, hydroxypropyl methylcellulose, hydroxypropylcellulose, hydroxypropyl methylcellulose phthalate, hydroxypropyl methylcellulose acetate succinate, and polyethylene glycol. More preferably, the water-soluble polymer includes at least one (C3) selected from the group consisting of polyvinylpyrrolidone, hydroxypropyl methylcellulose, hydroxypropylcellulose, and polyethylene glycol. Relative to 100 mass % of the water-soluble polymer, the content of the at least one (C2) or (C3) is preferably 70 to 100 mass %, more preferably 80 to 100 mass %, more preferably 90 to 100 mass %, still more preferably 95 to 100 mass %.

The molecular weight of the water-soluble polymer is not particularly limited; for example, the weight-average molecular weight (Mw) may be 1000 to 600,000, may be 1000 to 100,000, may be 5000 to 80000, or may be 10000 to 60000. The weight-average molecular weight (Mw) is a value measured by the GPC method.

For the water-soluble polymer, the K value is not particularly limited, is preferably, for example, 5 to 100, preferably 10 to 70, preferably 15 to 50, preferably 15 to 35. The K value is a value indicating the magnitude of molecular weight determined by the Fikentscher's method, and can be determined by the publicly known measurement method and the following Fikentscher's equation:

$$K value = \{ {[ 300 C log Z + {( C + 1.5 ClogZ )}^{2} ]}^{1 / 2} + 1.5 ClogZ - C \} / ( 0.15 C + 0.003 {C}^{2} )$$

where C represents the concentration (mass %) of the sample, the K value of PVP in EXAMPLES below was measured at a sample concentration of 1 mass %; Z represents the relative viscosity (ηrel) of the solution having the concentration C; the relative viscosity ηrel is determined by the following equation:

$$η rel = ( Flow time of solution ) / ( Flow time of water )$$

In one embodiment, the water-soluble polymer employed may be at least one N-vinyllactam-based polymer selected from the group consisting of homopolymers of N-vinyllactams and copolymers of N-vinyllactams. In this case, such an N-vinyllactam-based polymer employed preferably has a K value of 5 to 100, more preferably 10 to 70, more preferably 15 to 50, more preferably 15 to 35.

In one embodiment, the water-soluble polymer employed may be a hydroxyalkyl alkylcellulose such as HPMC. In this case, in the hydroxyalkyl alkylcellulose employed, preferably, the degree of substitution by hydroxyalkoxy groups (in the case of HPMC, hydroxypropoxy groups) is preferably 3 to 20 mass % (more preferably 5 to 15 mass %, more preferably 6 to 13 mass %, more preferably 6 to 12 mass %), and the degree of substitution by alkoxy groups (in the case of HPMC, methoxy groups) is 18 to 35 mass % (more preferably 20 to 33 mass %, more preferably 27 to 31 mass %, more preferably 27 to 30 mass %). The degrees of substitution are measured by a method according to the measurement method using a gas chromatograph described in Methylcellulose, Hydroxypropyl methylcellulose, and Hydroxypropylcellulose in Japan's Specifications and Standards for Food Additives, 9th edition.

The hydroxyalkyl alkylcellulose employed preferably has, in the case of being prepared as a 2 mass % aqueous solution, a viscosity (20° C.) of 1 to 100 mm²/s. More preferably, the hydroxyalkyl alkylcellulose employed has, in the case of being prepared as a 2 mass % aqueous solution, a viscosity (20° C.) of 1 to 20 mm²/s. The viscosity is more preferably 2 to 10 mm²/s, more preferably 2 to 7 mm²/s, or may be 2 to 5 mm²/s. The viscosity is measured in accordance with Viscosity Measurement by Capillary Tube Viscometer in Japan's Specifications and Standards for Food Additives, 9th edition.

In one embodiment, the water-soluble polymer employed may be a hydroxyalkylcellulose such as HPC. In this case, the hydroxyalkylcellulose has, in the case of being prepared as a 2 mass % aqueous solution, a viscosity (20° C.) of preferably 0.1 to 10000 mPa-s, more preferably 0.3 to 5000 mPa-s, more preferably 0.3 to 3000 mPa·s, more preferably 0.3 to 1000 mPa-s, more preferably 0.5 to 500 mPa·s, more preferably 0.8 to 100 mPa·s, more preferably 1 to 20 mPa·s, more preferably 1.2 to 10 mPa-s, more preferably 1.5 to 8 mPa·s, more preferably 1.8 to 4 mPa·s. The viscosity is measured in accordance with Viscosity Measurement by Rotational Viscometer in Japan's Specifications and Standards for Food Additives, 9th edition.

The hydroxyalkylcellulose has a weight-average molecular weight (Mw) of 1000 to 600,000, more preferably 2000 to 500,000, more preferably 3000 to 400,000, more preferably 5000 to 300,000, more preferably 10000 to 200,000, more preferably 15000 to 150,000, more preferably 20000 to 100,000, more preferably 25000 to 80000, more preferably 30000 to 50000. The weight-average molecular weight (Mw) is a value measured by the GPC method.

In the hydroxyalkylcellulose, the degree of substitution by hydroxyalkoxy groups (in the case of HPC, hydroxypropoxy groups) is preferably 5 to 99 mass %, more preferably 10 to 95 mass %, more preferably 15 to 90 mass %, more preferably 20 to 85 mass %. The degree of substitution is measured by a method according to the measurement method using a gas chromatograph described in Hydroxypropylcellulose in Japan's Specifications and Standards for Food Additives, 9th edition.

In one embodiment, the water-soluble polymer employed may be a polyalkylene glycol such as PEG and/or polyalkylene oxide. In this case, the polyalkylene glycol and/or polyalkylene oxide preferably has a viscosity (210° F.) of 10 to 100000 mm²/s, more preferably 15 to 50000 mm²/s, more preferably 20 to 30000 mm²/s, more preferably 50 to 20000 mm²/s, more preferably 100 to 10000 mm²/s, more preferably 200 to 5000 mm²/s, more preferably 400 to 3000 mm²/s, more preferably 600 to 1000 mm²/s. The viscosity is measured in accordance with Viscosity measurement by capillary tube viscometer in General Tests, Processes and Apparatus in the Japanese Pharmacopoeia 18th Edition.

The polyalkylene glycol and/or polyalkylene oxide preferably has a freezing point of 10 to 100° C., more preferably 20 to 90° C., more preferably 30 to 80° C., more preferably 40 to 70° C., more preferably 50 to 60° C. The freezing point is measured in accordance with General Tests, Processes and Apparatus in the Japanese Pharmacopoeia 18th Edition.

The polyalkylene glycol and/or polyalkylene oxide preferably has a number-average molecular weight (Mn) of 100 to 100000, more preferably 500 to 80000, more preferably 1000 to 50000, more preferably 2000 to 30000, more preferably 3000 to 20000, more preferably 6000 to 15000, more preferably 7000 to 10000. The number-average molecular weight of the polyalkylene glycol and/or polyalkylene oxide is calculated on the basis of the hydroxyl value measured in accordance with JIS K1557-1:2007.

### [Solid Composition]

The solid composition according to the embodiment includes the cannabinoid that has been amorphized using the sucrose fatty acid ester and the water-soluble polymer. Specifically, the water-soluble polymer and the sucrose fatty acid ester serving as an emulsifier enter between molecules of the cannabinoid to eliminate crystallinity of the cannabinoid to amorphize the cannabinoid. The cannabinoid having been thus amorphized is dispersed at a molecular level in the water-soluble polymer serving as an inactive carrier. Thus, in one embodiment, the solid composition is a solid dispersion. In general, the solid dispersion in which a slightly water-soluble medicament has been amorphized and the solid slightly water-soluble medicament having the amorphous form is dispersed at a molecular level in the inactive carrier, has high solubility in water.

The solid composition according to the embodiment is solid at room temperature (25° C.) and more specifically remains solid even after being left at room temperature for 5 hours.

In the solid composition, a mass ratio (B)/(A), which is a ratio of the mass of the (B) sucrose fatty acid ester to the mass of the (A) cannabinoid, is preferably 0.2 to 30, more preferably 0.5 to 20, still more preferably 1.0 to 15, still more preferably 1.5 to 12, still more preferably 2.0 to 10, still more preferably 2.5 to 8.0, still more preferably 3.0 to 6.0.

In the solid composition, a mass ratio (C)/(A), which is a ratio of the mass of the (C) water-soluble polymer to the mass of the (A) cannabinoid, is preferably 0.2 to 30, more preferably 0.5 to 25, still more preferably 1.0 to 20, still more preferably 2.0 to 15, still more preferably 3.0 to 12, still more preferably 4.0 to 10, still more preferably 5.0 to 9.0.

In the solid composition, a mass ratio (C)/(B), which is a ratio of the mass of the (C) water-soluble polymer to the mass of the (B) sucrose fatty acid ester, is preferably 0.1 to 10, more preferably 0.2 to 8.0, more preferably 0.3 to 5.0, still more preferably 0.5 to 4.0, still more preferably 1.0 to 3.0, or may be 1.5 to 2.5.

The solid composition according to the embodiment may include, in addition to the components (A) to (C), another component. The other component is not particularly limited, and examples include excipients, binders, fillers, lubricants, extending agents, disintegrants, surfactants, seasonings, and flavors.

The form of the solid composition is not particularly limited, may be a powder or may be granules provided by subjecting the powder to granulation; the solid composition may have a form of various solid preparations.

### [Method for Producing Solid Composition]

The method for producing the solid composition according to the embodiment is not particularly limited. In one embodiment, the method for producing the solid composition includes the following steps:
(1) a step of dissolving a cannabinoid, a sucrose fatty acid ester having a monoester ratio of 85 mass % or more, and a water-soluble polymer in a solvent to obtain a solution, and
(2) a step of removing the solvent from the obtained solution.

In the step (1), the solvent employed is a solvent that can dissolve the cannabinoid, the sucrose fatty acid ester, and the water-soluble polymer and is not particularly limited. Specific examples of the solvent include alcohols such as methanol, ethanol, 1-propanol, isopropanol, 1-butanol, and 2-butanol, ketones such as methyl ethyl ketone and acetone, acetates such as ethyl acetate and methyl acetate, ethers such as diethyl ether, alkanes such as propane, butane, and hexane, polyhydric alcohols such as propylene glycol and glycerol, hydrocarbon chlorides such as dichloromethane, chloroform, and dichloroethane, and organic solvents such as cyclohexane, and one of the foregoing or a solvent mixture of two or more of the foregoing may be employed. Alternatively, a solvent mixture of such an organic solvent and water may be employed.

In the step (1), the solvent under heating is used to dissolve the cannabinoid, the sucrose fatty acid ester, and the water-soluble polymer. During this process, dissolution is preferably achieved by stirring or application of ultrasonic vibrations using an ultrasonic apparatus. The heating temperature for the solvent is not particularly limited, and is preferably, for example, 60 to 90° C.

In the solution prepared in the step (1), the concentrations of the components are not particularly limited. For example, the concentration of the cannabinoid may be 0.1 to 5 mass %, or may be 0.2 to 3 mass %. The concentrations of the sucrose fatty acid ester and the water-soluble polymer may be set in accordance with the mass ratios (B)/(A), (C)/(A), and (C)/(B) of the components in the solid composition to be produced.

In the step (2), the solvent is removed from the solution obtained in the step (1) to thereby obtain the solid composition. The method for removing the solvent is not particularly limited, and examples include vacuum drying, spray drying, freeze drying, heat drying, and natural drying. After the solvent is removed, a grinding process may be performed.

### [Applications of Solid Composition]

The solid composition according to the embodiment is suitably used in applications such as pharmaceuticals, quasi-drugs, foods such as functional health foods (such as foods for specified health uses, foods with nutrient function claims, and foods with function claims), health foods, dietary supplements, and supplements, pet foods, and cosmetics. The solid composition is preferably used as oral preparations. Thus, an oral composition according to one preferred embodiment includes the above-described solid composition.

The oral composition may be constituted only by the solid composition, but may include, together with the solid composition, another food material, another active component, and/or an additive, for example. Examples of the additive, include excipients, binders, fillers, lubricants, extending agents, disintegrants, surfactants, seasonings, flavors, and coloring agents.

The form of the oral composition is not particularly limited, and examples include tablets, granules, powders, fine granules, granules, pills, and capsules.

The solid composition according to the embodiment provides high water-solubility of the cannabinoid, hence, in the case of oral administration or ingestion, is expected to exhibit a high degree of dissolution of the cannabinoid into the body fluid, and enables efficient ingestion of the cannabinoid.

### EXAMPLES

Hereinafter, the present invention will be described further in detail on the basis of Examples and Comparative Examples; however, the present invention is not limited to this.

### Examples 1 to 4 and Comparative Examples 1 to 6

In accordance with a formula (parts by mass) in Table 1 below, the (A) cannabinoid, the (B) sucrose fatty acid ester, polyvinylpyrrolidone (PVP) serving as the (C) water-soluble polymer, and the solvent in a total amount of 20 g were charged into a 50 mL screw vial.

Subsequently, (1) a heating process of immersing the screw vial in a hot water bath at 75° C. for 4 minutes, and (2) an ultrasonic process of immersing the screw vial in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabinoid, the sucrose fatty acid ester, and the vinylpyrrolidone were completely dissolved.

From the resultant solution, the solvent was distilled off using an evaporator. In the process using the evaporator, the pressure was reduced at 75° C. from 400 hPa to 160 hPa to distill off the solvent almost completely; and subsequently the process was further continued at 40 to 60 hPa for 30 minutes.

The solid adhering to the wall surface of the recovery flask was scraped off with a spatula; the solid was ground in an agate mortar; in this way, solid compositions of Examples 1 to 4 and Comparative Examples 2 to 6 were obtained.

Details of the components in the Table are as follows.
CBD: cannabidiol, “CBD Isolate Powder” manufactured by COACH IND, INC.
SE-SS: “DK ESTER SS” manufactured by DAI-ICHI KOGYO SEIYAKU CO., LTD., a sucrose fatty acid ester in which a constituent fatty acid is stearic acid having 18 carbon atoms serving as the main component, the monoester ratio is 99 mass %, the ratio of the remaining diester and triester is 1 mass %, and HLB=19
SE-F160: “DK ESTER F-160” manufactured by DAI-ICHI KOGYO SEIYAKU CO., LTD., a sucrose fatty acid ester in which a constituent fatty acid is stearic acid having 18 carbon atoms serving as the main component, the monoester ratio is 70 mass %, the ratio of the remaining diester and triester is 30 mass %, and HLB=15
SE-F50: “DK ESTER F-50” manufactured by DAI-ICHI KOGYO SEIYAKU CO., LTD., a sucrose fatty acid ester in which a constituent fatty acid is stearic acid having 18 carbon atoms serving as the main component, the monoester ratio is 30 mass %, the remaining di- or higher esters correspond to 70 mass %, and HLB=6
PVP: polyvinylpyrrolidone, “AIPHTACT K-30PH” (Mw=45,000, K value=30) manufactured by DAI-ICHI KOGYO SEIYAKU CO., LTD.

The obtained solid compositions were subjected to, as an amorphization evaluation, X-ray diffractometry (XRD). In addition, the solid compositions were subjected to, as a water-solubility evaluation, a dissolution test 1. Note that, in Comparative Example 1, the cannabidiol powder itself was used as the solid composition and subjected to the evaluations. Comparative Examples 4 and 5, which were not subjected to the amorphization evaluation, are described with “-” in Table 1.

### (Amorphization Evaluation: XRD)

A “RINT UltimaIII horizontal goniometer (D/teX-25)” manufactured by Rigaku Corporation was used to obtain the XRD chart of such a solid composition. The XRD measurement conditions were the focusing method, X-ray: Cu/40 kV/40 mA, scanning range: 3.0° to 50.0°, and scanning axis: 2θ/θ. The obtained XRD chart was used to evaluate amorphization of the cannabidiol in accordance with the following grades.
A: No peaks specific to cannabidiol are observed (amorphous form).
B: The peaks specific to cannabidiol are observed (crystalline cannabidiol is included).

### (Dissolution Test 1: Dissolution Amount)

As the dissolution testing apparatus, an “NTR-6600AST” manufactured by Toyama Sangyo Co., Ltd. was used; the vessel was charged with 900 mL of ion-exchanged water; for the purpose of degassing, the water was stirred overnight at a water temperature of 37° C.±0.2° C. A gelatin capsule (“HF capsule” manufactured by Matsuya Corporation) filled with the solid composition containing 8 mg of the cannabidiol was placed into a sinker, and immersed into the vessel. After a lapse of a predetermined time, sampling was performed in the vessel and the sample was filtered through a 0.45 μm membrane filter. This procedure was performed individually for such predetermined times of 60 minutes and 120 minutes. The filtrate was diluted two-fold with ethanol and filtered through a 0.20 μm membrane filter; HPLC was performed to quantify the cannabidiol. For each solid composition, two samples were tested and the resultant values were averaged; the average was rounded to two decimal places to determine the dissolution amount.

The 0.45 μm membrane filter employed was “25HP045AN” (filter material: PTFE, pore size: 0.45 μm) manufactured by ADVANTEC. The 0.20 μm membrane filter employed was “13HP020AN” (filter material: PTFE, pore size: 0.20 μm) manufactured by ADVANTEC.

The HPLC conditions were column: octadecylsilyl column, solvent: mixed solution of acetonitrile/10 mM aqueous ammonium acetate solution, and wavelength: 210 nm.

TABLE 1

| | | | | | | | Compar- | Compar- | Compar- | Compar- | Compar- | Compar- |
|---|---|---|---|---|---|---|---|---|---|---|---|---|
| | | | | | | | ative | ative | ative | ative | ative | ative |
| | | | Exam- | Exam- | Exam- | Exam- | Exam- | Exam- | Exam- | Exam- | Exam- | Exam- |
| | | | ple 1 | ple 2 | ple 3 | ple 4 | ple 1 | ple 2 | ple 3 | ple 4 | ple 5 | ple 6 |
| Formula | (A) | CBD | 1 | 1 | 1 | 1 | 1 | 1 | 1 | 1 | 1 | 1 |
| [parts | (B) | SE-SS | 2 | 4 | 4 | 8 | — | 4 | — | — | — | — |
| by mass] | | SE-F160 | — | — | — | — | — | — | — | 2 | — | — |
| | | SE-F50 | — | — | — | — | — | — | — | — | 2 | 4 |
| | (C) | PVP | 4 | 4 | 8 | 8 | — | — | 8 | 4 | 4 | 8 |
| | Solvent | Ethanol | 93 | 91 | 87 | 83 | — | 95 | 91 | 93 | 93 | 87 |
| Mass ratio | (B)/(A) | | 2.0 | 4.0 | 4.0 | 8.0 | 0.0 | 4.0 | 0.0 | 2.0 | 2.0 | 4.0 |
| | (C)/(A) | | 4.0 | 4.0 | 8.0 | 8.0 | 0.0 | 0.0 | 8.0 | 4.0 | 4.0 | 8.0 |
| | (C)/(B) | | 2.0 | 1.0 | 2.0 | 1.0 | — | 0.0 | — | 2.0 | 2.0 | 2.0 |
| Evalu- | Amorphization | | A | A | A | A | B | B | A | — | — | A |
| ations | evaluation XRD | | | | | | | | | | | |
| | Disso- | After | 0.44 | 0.06 | 3.17 | 0.07 | Less | 0.03 | 0.01 | Less | Less | 0.01 |
| | lution | lapse of | | | | | than 0.01 | | | than 0.01 | than 0.01 | |
| | amount | 60 minutes | | | | | | | | | | |
| | [μg/mL] | After | 0.64 | 0.46 | 4.08 | 0.88 | 0.01 | 0.04 | 0.01 | Less | Less | 0.01 |
| | | lapse of | | | | | | | | than 0.01 | than 0.01 | |
| | | 120 minutes | | | | | | | | | | |

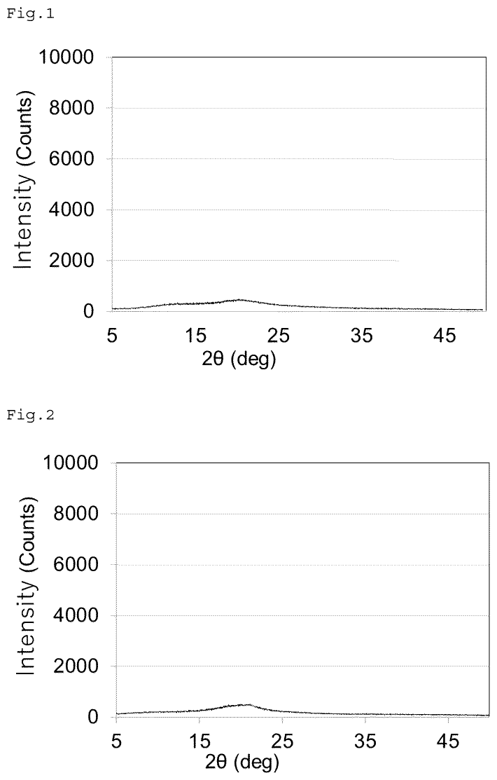

The results are described in Table 1 and FIGS. 1 and 4 to 6. Comparative Example 1 is an example using the cannabidiol powder and, as illustrated in FIG. 4, the XRD chart had the peaks specific to the cannabidiol. Thus, the cannabidiol was in the crystalline state and, in the dissolution test 1, the dissolution amount was less than the detection limit value after the lapse of 60 minutes, and 0.01 μg/mL even after the lapse of 120 minutes; the cannabidiol substantially did not dissolve in water.

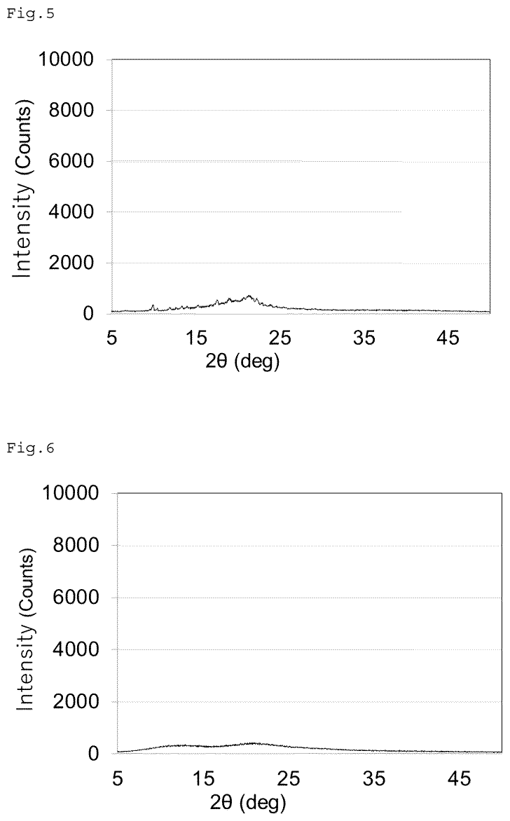

In Comparative Example 2, the solid composition was prepared by dissolving the cannabidiol together with the sucrose fatty acid ester in ethanol. In Comparative Example 2, as illustrated in FIG. 5, a portion of the cannabidiol was amorphized; but the water-soluble polymer was not additionally used for the preparation, hence the peaks specific to the cannabidiol remained and the amorphization was insufficient. In addition, in Comparative Example 2, the effect of improving the solubility in water was also weak.

In Comparative Example 3, the solid composition was prepared by dissolving the cannabidiol together with PVP in ethanol. In Comparative Example 3, as illustrated in FIG. 6, the peaks specific to the cannabidiol were not observed and the cannabidiol was amorphized. However, in the dissolution test 1, the effect of improving the solubility in water was substantially not observed.

In Comparative Examples 4 to 6, the solid compositions were prepared using PVP together with the sucrose fatty acid esters, but the sucrose fatty acid esters employed had a monoester ratio of 70 mass % or 30 mass %. As described in Table 1, in Comparative Example 6, the cannabidiol had the amorphous form. Similarly, in Comparative Examples 4 and 5, the cannabidiols inferentially had the amorphous form (not measured). However, in Comparative Examples 4 to 6, the sucrose fatty acid esters employed had low monoester ratios and the effect of improving the solubility in water was poor.

In Examples 1 to 4, the solid compositions were prepared using the sucrose fatty acid ester having the high monoester ratio together with PVP. As illustrated in FIG. 1, in Example 3, the XRD chart had no peaks specific to the cannabidiol and the cannabidiol had the amorphous form. Also for Examples 1, 2, and 4, similar XRD charts were obtained and the cannabidiols had the amorphous form. Thus, in Examples 1 to 4, solid dispersions including amorphous cannabidiols were obtained. In the dissolution test 1, in Examples 1 to 4, as described in Table 1, the solubility in water was remarkably improved, compared with Comparative Example 1; in particular, Example 3 provided strongly the improvement effect.

### [Oral Absorption Evaluation: Animal Experiment]

The solid composition of Example 3, which was found to exhibit the particularly strong effect of improving the solubility in water, was used in an animal experiment to evaluate the oral absorption. For comparison, the powder composed only of cannabidiol in Comparative Example 1 was similarly subjected to an animal experiment. The evaluation method was as follows.

### (Oral Absorption Evaluation Method)

Three male SD rats were subjected to oral administration at an administration amount of 30 mg/kg in terms of cannabidiol; after the administration, blood samples were taken after the lapse of 0.5 hours, 1 hour, 1.5 hours, 2 hours, 3 hours, 4 hours, 8 hours, and 24 hours; the cannabidiol concentration in the blood was measured by liquid chromatograph-mass spectrometry (LC-MS/MS), and AUC (area under the blood concentration time curve) of the cannabidiol was determined. Of the three rats, data of the highest AUC was employed.

In the LC-MS/MS measurement, as a pre-treatment, 20 μL of the plasma sample and 80 μL of methanol were placed into an Eppendorf tube and suspended for deproteinization. After the proteins were precipitated in a centrifuge, the supernatant was subjected to centrifugal filtration using a filter (manufactured by Millipore, Ultrafree-MC, Hydrophilic PTFE membrane, 0.2 μm). This solution and ultrapure water were mixed together in 1:1 (mass ratio), and used as the measurement sample. The measurement conditions of LC-MS/MS were column: octadecylsilyl column, ion source: ESI+, mobile phase: solution mixture of methanol/10 mM aqueous ammonium formate solution.

As a result, the AUC of the cannabidiol was 812 h·ng/mL in Comparative Example 1 whereas the AUC of the cannabidiol was 1906 h·ng/mL in Example 3, which has been demonstrated to exhibit high absorption efficiency by oral ingestion into the body.

### Example 5

A 1 L medium bottle was charged with 1 part by mass of an (A) cannabidiol, 4 parts by mass of a (B) sucrose fatty acid ester, and 43.5 parts by mass of ethanol. The cannabidiol employed was CBD of Example 1 and the sucrose fatty acid ester employed was SE-SS of Example 1.

Subsequently, (1) a heating process of immersing the medium bottle in a hot water bath at 75° C. for 4 minutes, and (2) an ultrasonic process of immersing the medium bottle in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabidiol and the sucrose fatty acid ester were completely dissolved.

Subsequently, the medium bottle was charged with 8 parts by mass of HPMC-1 serving as the water-soluble polymer (C) and 43.5 parts by mass of methylene chloride; a compact ultrasonic apparatus (“BRANSON 2210” manufactured by Yamato Scientific Co., Ltd.) was used to radiate ultrasonic waves in a bath containing water at room temperature (in the apparatus, “SET SONICS min” was selected and turned it ON) to achieve dissolution.

The HPMC-1 was hydroxypropyl methylcellulose “METOLOSE SE-03” manufactured by Shin-Etsu Chemical Co., Ltd. (viscosity: 3 mm²/s (20° C., 2 mass % aqueous solution), degree of substitution: 28.8 mass % for methoxy groups and 8.7 mass % for hydroxypropoxy groups).

The resultant solution was dried using a spray dryer to provide the solid composition of Example 5. The spray dryer employed was “ADL311S-A” manufactured by Yamato Scientific Co., Ltd. and the solvent recovery apparatus employed was “GAS410” manufactured by Yamato Scientific Co., Ltd. The drying conditions were an inlet set temperature of 55° C. and an outlet set temperature of 39° C.

The solid composition of Example 5 was subjected to, as in Example 1, X-ray diffractometry (XRD) serving as the amorphization evaluation. As a result, as illustrated in FIG. 2, the XRD chart had no peaks specific to the cannabidiol and the cannabidiol had the amorphous form. Thus, the solid composition of Example 5 has been demonstrated to be a solid dispersion including an amorphous cannabidiol.

The solid composition of Example 5 was subjected to, as a water-solubility evaluation, a dissolution test 2 below. The results are as described in Table 2 below and the dissolution amount of Example 5 was 5.33 μg/mL. As a control, the solid composition of Comparative Example 1 was similarly subjected to the dissolution test 2, and the dissolution amount was 0.03 μg/mL. Thus, in Example 5, the solubility in water was remarkably improved, compared with Comparative Example 1.

### (Dissolution Test 2)

In a 50 mL screw vial, such a solid composition including 0.4 mg of cannabidiol was dissolved in 45 g of ion-exchanged water. The resultant solution was filtered through a 0.45 μm membrane filter; the filtrate was diluted two-fold with ethanol and further filtered through a 0.20 μm membrane filter; HPLC was performed to quantify the cannabidiol. The value was rounded to two decimal places to determine the dissolution amount. The 0.45 μm membrane filter, the 0.20 μm membrane filter, and the HPLC conditions were as described in the dissolution test 1.

### Example 6

The same procedures as in Example 5 were performed except that, as the water-soluble polymer (C), 8 parts by mass of HPMC-1 was replaced by 8 parts by mass of HPMC-2, to obtain the solid composition of Example 6.

The HPMC-2 is hydroxypropyl methylcellulose manufactured by Shin-Etsu Chemical Co., Ltd. “METOLOSE SE-06” (viscosity: 6 mm²/s (20° C., 2 mass % aqueous solution), degrees of substitution: 28.5 mass % for methoxy groups, 8.7 mass % for hydroxypropoxy groups).

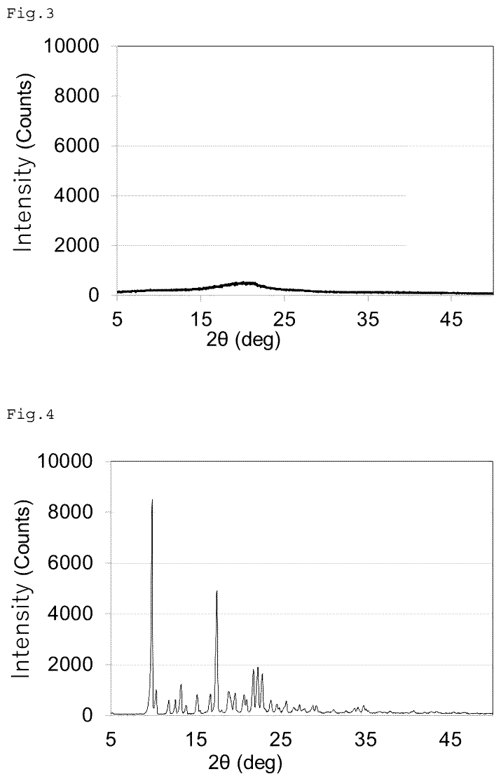

The solid composition of Example 6 was subjected to, as in Example 1, X-ray diffractometry (XRD) serving as the amorphization evaluation; as a result, as illustrated in FIG. 3, the XRD chart had no peaks specific to the cannabidiol and the cannabidiol had the amorphous form. Thus, the solid composition of Example 6 has been demonstrated to be a solid dispersion including an amorphous cannabidiol.

The solid composition of Example 6 was subjected to evaluation of the dissolution test 2; as a result, as described in Table 2, the dissolution amount was 4.04 μg/mL and the solubility in water was remarkably improved, compared with the solid composition of Comparative Example 1 serving as the control.

TABLE 2

| | | | Example 5 | Example 6 | Comparative Example 1 |
|---|---|---|---|---|---|
| Formula | (A) | CBD | 1 | 1 | 1 |
| [parts by mass] | (B) | SE-SS | 4 | 4 | — |
| | (C) | HPMC-1 | 8 | — | — |
| | | HPMC-2 | — | 8 | — |
| | Solvent | Ethanol | 43.5 | 43.5 | — |
| | | Methylene chloride | 43.5 | 43.5 | — |
| Mass ratio | (B)/(A) | | 4.0 | 4.0 | 0.0 |
| | (C)/(A) | | 8.0 | 8.0 | 0.0 |
| | (C)/(B) | | 2.0 | 2.0 | — |
| Evaluations | Amorphization evaluation XRD | | A | A | B |
| | Dissolution test 2 | Dissolution amount [μg/mL] | 5.33 | 4.04 | 0.03 |

### [Oral Absorption Evaluation of Solid Composition of Example 5]

The solid composition of Example 5 was evaluated for oral absorption by an animal experiment similar to that for the solid composition of Example 3. Specifically, while the powder composed only of cannabidiol in Comparative Example 1 was used as the control, the animal experiment was performed in accordance with the above-described oral absorption evaluation method.

As a result, the AUC of the cannabidiol was 665 h·ng/mL in Comparative Example 1 whereas the AUC of the cannabidiol was 1487 h·ng/mL in Example 5, which has been demonstrated to provide higher absorption efficiency by oral ingestion into the body.

### Examples 7 to 10 and Comparative Examples 7 to 8

In accordance with a formula (parts by mass) in Table 3 below, the (A) cannabidiol (CBD), the (B) sucrose fatty acid ester, hydroxypropylcellulose (HPC) serving as the water-soluble polymer (C), and the solvent in a total amount of 40 g were prepared. Of these, the cannabidiol, the sucrose fatty acid ester, and the solvent were charged into a 50 mL screw vial; subsequently, (1) a heating process of immersing the screw vial in a hot water bath at 75° C. for 4 minutes, and (2) an ultrasonic process of immersing the screw vial in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabidiol and the sucrose fatty acid ester were completely dissolved.

To the resultant solution being stirred using a stirring bar, hydroxypropylcellulose was added and stirred until it was dissolved. Note that, when it was not dissolved, it was subjected to ultrasonic waves at 20 to 40° C. to thereby be dissolved. Subsequently, an evaporator was used to distill off the solvent. In the process using the evaporator, the pressure was reduced at 75° C. from 400 hPa to 160 hPa, to distill off the solvent almost completely; and subsequently the process was further continued at 40 to 60 hPa for 40 minutes. The solid adhering to the wall surface of the recovery flask was scraped off with a spatula; the solid was ground in an agate mortar; in this way, the solid compositions of Examples 7 to 10 and Comparative Examples 7 to 8 were obtained.

### Examples 11 to 13 and Comparative Examples 9 to 10

In accordance with a formula (parts by mass) in Table 3 below, the (A) cannabidiol (CBD), the (B) sucrose fatty acid ester, polyethylene glycol (PEG) serving as the (C) water-soluble polymer, and the solvent in a total amount of 40 g were charged into a 50 mL screw vial. Subsequently, (1) a heating process of immersing the screw vial in a hot water bath at 75° C. for 4 minutes, and (2) an ultrasonic process of immersing the screw vial in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabidiol, the sucrose fatty acid ester, and the polyethylene glycol were completely dissolved.

For the resultant solution, an evaporator was used to distill off the solvent. In the process using the evaporator, the pressure was reduced at 75° C. from 400 hPa to 160 hPa, to distill off the solvent almost completely; and subsequently the process was further continued at 10 to 60 hPa for 40 minutes. The solid adhering to the wall surface of the recovery flask was scraped off with a spatula; in this way, the solid compositions of Examples 11 to 13 and Comparative Examples 9 to 10 were obtained.

For the components in Table 3, CBD and SE-SS are the same as in Table 1, and HPC and PEG are as follows.
HPC: hydroxypropylcellulose “CELNY SSL” manufactured by Nippon Soda Co., Ltd. (viscosity: 2.0 to 2.9 mPa·s (20° C., 2 mass % aqueous solution), weight-average molecular weight: 40000)
PEG: “MACROGOL 6000 (SP)” manufactured by Sanyo Chemical Industries, Ltd. (viscosity: 800 mm²/s (210° F.), number-average molecular weight: 8600)

For the obtained solid compositions, Examples 7 to 10 and Comparative Examples 7 to 8 were subjected to the above-described X-ray diffractometry (XRD) serving as the amorphization evaluation. For Examples 11 to 13, PEG used as the water-soluble polymer has crystallinity, which hampers the evaluation by XRD; thus, differential scanning calorimetry (DSC) described below was performed. Note that, for Comparative Examples 9 to 10, the amorphization evaluation was not performed.

### (Amorphization Evaluation: DSC)

A thermal analysis apparatus “Thermo Plus EVO DSC 8230” manufactured by Rigaku Corporation was used to obtain a DSC chart of such a solid composition. The measurement conditions of DSC were set such that reference substance: Al, measurement atmosphere: N₂ 40 mL/min, heating rate: 10.0° C./min, and temperature range: 25 to 205° C. The obtained DSC chart was used to evaluate whether or not the cannabidiol was amorphized, in accordance with the following grades.
A: No melting point peak of the cannabidiol is observed (amorphous state)
B: The melting point peak of the cannabidiol is observed (a crystalline cannabidiol is included)

The solid compositions of Examples 7 to 13 and Comparative Examples 7 to 10 were subjected to the above-described dissolution test 1 serving as the water-solubility evaluation. Note that the predetermined times in the dissolution test 1 were set at 60 minutes, 120 minutes, and 240 minutes. The solid composition of Comparative Example 1 serving as the control was similarly subjected to the dissolution test 1.

TABLE 3

| | | | Exam- | Exam- | Exam- | Exam- | Exam- | Exam- | Exam- |
|---|---|---|---|---|---|---|---|---|---|
| | | | ple 7 | ple 8 | ple 9 | ple 10 | ple 11 | ple 12 | ple 13 |
| Formula | (A) | CBD | 1 | 1 | 1 | 1 | 1 | 1 | 1 |
| [parts | (B) | SE-SS | 2 | 4 | 4 | 8 | 4 | 4 | 8 |
| by mass] | (C) | HPC | 4 | 4 | 8 | 8 | — | — | — |
| | | PEG | — | — | — | — | 4 | 8 | 8 |
| | Solvent | Ethanol | 93 | 91 | 87 | 83 | 91 | 87 | 83 |
| Mass ratio | (B)/(A) | | 2.0 | 4.0 | 4.0 | 8.0 | 4.0 | 4.0 | 8.0 |
| | (C)/(A) | | 4.0 | 4.0 | 8.0 | 8.0 | 4.0 | 8.0 | 8.0 |
| | (C)/(B) | | 2.0 | 1.0 | 2.0 | 1.0 | 1.0 | 2.0 | 1.0 |
| Evalu- | Amorphization | XRD | A | A | A | A | — | — | — |
| ations | evaluation | DSC | — | — | — | — | A | A | A |
| | Disso- | After | 0.02 | 0.01 | Less | 0.16 | 0.03 | 0.25 | 1.04 |
| | lution | lapse of | | | than 0.01 | | | | |
| | amount | 60 minutes | | | | | | | |
| | [μg/mL] | After | 0.06 | 0.10 | 0.09 | 0.77 | 0.14 | 0.52 | 1.37 |
| | | lapse of | | | | | | | |
| | | 120 minutes | | | | | | | |
| | | After | 0.17 | 0.38 | 0.34 | 2.28 | 0.31 | 0.88 | 1.97 |
| | | lapse of | | | | | | | |
| | | 240 minutes | | | | | | | |

| | | | Compar- | Compar- | Compar- | Compar- | Compar- |
|---|---|---|---|---|---|---|---|
| | | | ative | ative | ative | ative | ative |
| | | | Exam- | Exam- | Exam- | Exam- | Exam- |
| | | | ple 1 | ple 7 | ple 8 | ple 9 | ple 10 |
| Formula | (A) | CBD | 1 | 1 | 1 | 1 | 1 |
| [parts | (B) | SE-SS | — | — | — | — | — |
| by mass] | (C) | HPC | — | 4 | 8 | — | — |
| | | PEG | — | — | — | 4 | 8 |
| | Solvent | Ethanol | — | 95 | 91 | 95 | 91 |
| Mass ratio | (B)/(A) | | 0.0 | 0.0 | 0.0 | 0.0 | 0.0 |
| | (C)/(A) | | 0.0 | 4.0 | 8.0 | 4.0 | 8.0 |
| | (C)/(B) | | — | — | — | — | — |
| Evalu- | Amorphization | XRD | B | A | A | — | — |
| ations | evaluation | DSC | — | — | — | — | — |
| | Disso- | After | Less | Less | Less | 0.03 | 0.03 |
| | lution | lapse of | than 0.01 | than 0.01 | than 0.01 | | |
| | amount | 60 minutes | | | | | |
| | [μg/mL] | After | 0.01 | 0.02 | 0.01 | 0.03 | 0.04 |
| | | lapse of | | | | | |
| | | 120 minutes | | | | | |
| | | After | 0.01 | 0.01 | 0.02 | 0.03 | 0.04 |
| | | lapse of | | | | | |
| | | 240 minutes | | | | | |

The results are as described in Table 3; even in the cases of using, as the water-soluble polymer, hydroxypropylcellulose or polyethylene glycol, in Examples 7 to 13, the cannabidiol had the amorphous form and solid dispersions were obtained. In addition, the sucrose fatty acid ester having the high monoester ratio was used to thereby provide remarkably improved solubility in water, compared with Comparative Example 1. On the other hand, in Comparative Examples 7 to 10, sucrose fatty acid esters having a high monoester ratio were not used and the effect of improving the solubility in water was poor.

### Examples 14 to 15

In accordance with a formula (parts by mass) in Table 4 below, cannabigerol (CBG) serving as the (A) cannabinoid, the (B) sucrose fatty acid ester, polyvinylpyrrolidone (PVP) serving as the (C) water-soluble polymer, and the solvent in a total amount of 40 g were charged into a 50 mL screw vial. Subsequently, (1) a heating process of immersing the screw vial in a hot water bath at 75° C. for 4 minutes, and (2) an ultrasonic process of immersing the screw vial in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabigerol, the sucrose fatty acid ester, and the vinylpyrrolidone were completely dissolved.

For the resultant solution, an evaporator was used to distill off the solvent. In the process using the evaporator, the pressure was reduced at 75° C. from 400 hPa to 160 hPa, to distill off the solvent almost completely; and subsequently the process was further continued at 10 to 60 hPa for 30 minutes. The solid adhering to the wall surface of the recovery flask was scraped off with a spatula and the solid was ground in an agate mortar, to obtain the solid compositions of Examples 14 to 15.

### Example 16

In accordance with the formula (parts by mass) in Table 4 below, the (A) cannabigerol (CBG), the (B) sucrose fatty acid ester, hydroxypropylcellulose (HPC) serving as the (C) water-soluble polymer, and the solvent in a total amount of 40 g were prepared. Of these, the cannabigerol, the sucrose fatty acid ester, and the solvent were charged into a 50 mL screw vial; subsequently, (1) a heating process of immersing the screw vial in a hot water bath at 75° C. for 4 minutes, and (2) an ultrasonic process of immersing the screw vial in the bath at 50° C. of a compact ultrasonic apparatus (“BRANSON 2210” manufactured by Yamato Scientific Co., Ltd.) for 4 minutes (in the apparatus, “SET SONICS min” was selected and turned it ON) were performed; (1) and (2) above were repeated until the cannabigerol and the sucrose fatty acid ester were completely dissolved.

To the resultant solution being stirred using a stirring bar, hydroxypropylcellulose was added and stirred until it was dissolved. Subsequently, an evaporator was used to distill off the solvent. In the process using the evaporator, the pressure was reduced at 75° C. from 400 hPa to 160 hPa, to distill off the solvent almost completely; and subsequently the process was further continued at 5 to 60 hPa for 40 minutes. The solid adhering to the wall surface of the recovery flask was scraped off with a spatula, and the solid was ground in an agate mortar, to obtain the solid composition of Example 16. Note that, for the components in Table 4, SE-SS, PVP, and HPC are the same as in Table 1 and Table 3.

The solid compositions of Examples 14 to 16 were subjected to, as in Example 1, X-ray diffractometry (XRD) serving as the amorphization evaluation. In addition, as the evaluation for the solubility in water, the above-described dissolution test 1 was performed. Note that the predetermined times in the dissolution test 1 were set at 60 minutes, 120 minutes, and 240 minutes.

TABLE 4

| | | | Example 14 | Example 15 | Example 16 |
|---|---|---|---|---|---|
| Formula | (A) | CBG | 1 | 1 | 1 |
| [parts by mass] | (B) | SE-SS | 4 | 8 | 8 |
| | (C) | PVP | 8 | 8 | — |
| | | HPC | — | — | 8 |
| | Solvent | Ethanol | 87 | 83 | 83 |
| Mass ratio | (B)/(A) | | 4.0 | 8.0 | 8.0 |
| | (C)/(A) | | 8.0 | 8.0 | 8.0 |
| | (C)/(B) | | 2.0 | 1.0 | 1.0 |
| Evaluations | Amorphization evaluation XRD | | A | A | A |
| | Dissolution amount | After lapse of 60 minutes | 4.78 | 0.15 | 0.23 |
| | [μg/mL] | After lapse of | 4.73 | 0.38 | 0.71 |
| | | 120 minutes | | | |
| | | After lapse of | 4.34 | 0.72 | 1.76 |
| | | 240 minutes | | | |

The results are as described in Table 4; even in the cases of using, as the cannabinoid, cannabigerol, in Examples 14 to 16, the cannabigerol had the amorphous form and solid dispersions were obtained. In addition, the sucrose fatty acid ester having the high monoester ratio was used to thereby provide high solubility in water.

Note that, for various numerical ranges described in the Description, the upper limit values and the lower limit values can be appropriately combined, and all such combinations are regarded as being described as preferred numerical ranges in the Description. The numerical ranges described in the form of “X to Y” mean X or more and Y or less.

Some embodiments of the present invention have been described so far. These embodiments are described as examples and are not intended to limit the scope of the invention. These embodiments can also be carried out in various other forms and various omissions, replacements, and changes can be performed without departing from the spirit and scope of the invention. These embodiments and their omissions, replacements, changes, and the like are included in the scope and spirit of the invention and similarly included in the invention described in Claims and the range of equivalents.

1. A solid composition comprising:
an amorphous cannabinoid;
a sucrose fatty acid ester having a monoester ratio of 85 mass % or more; and
a water-soluble polymer.

2. The solid composition according to claim 1, wherein a mass ratio of the sucrose fatty acid ester to the cannabinoid is 0.2 to 30.

3. The solid composition according to claim 1, wherein a mass ratio of the water-soluble polymer to the cannabinoid is 0.2 to 30.

4. The solid composition according to claim 1, wherein a mass ratio of the water-soluble polymer to the sucrose fatty acid ester is 0.1 to 10.

5. The solid composition according to claim 1, wherein the sucrose fatty acid ester includes a fatty acid having 12 to 22 carbon atoms as a constituent fatty acid.

6. The solid composition according to claim 1, wherein the water-soluble polymer includes at least one selected from the group consisting of homopolymers of N-vinyllactams, copolymers of N-vinyllactams, cellulose ethers, cellulose esters, polyalkylene glycols, and polyalkylene oxides.

7. The solid composition according to claim 1, wherein the cannabinoid includes at least one selected from the group consisting of cannabidiol, cannabigerol, tetrahydrocannabinol, and cannabinol.

8. An oral composition comprising the solid composition according to claim 1.

9. A method for producing a solid composition, the method comprising:
dissolving a cannabinoid, a sucrose fatty acid ester having a monoester ratio of 85 mass % or more, and a water-soluble polymer in a solvent to obtain a solution; and
removing the solvent from the solution.
